(19) United States
(12) Patent Application Publication
(10) Pub. No.: US 2024/0416035 A1
(43) Pub. Date: Dec. 19, 2024

(54) Electronic Unit and Mobile Electronic Device for a Drug Delivery Device

(71) Applicant: Sanofi, Paris (FR)

(72) Inventors: Stefan Alt, Frankfurt am Main (DE); Michael Helmer, Frankfurt am Main (DE)

(21) Appl. No.: 18/705,843

(22) PCT Filed: Nov. 1, 2022
(86) PCT No.: PCT/EP2022/080402

§ 371 Date: Apr. 29, 2024

(30) Foreign Application Priority Data
Nov. 3, 2021 (EP) 21315213.5

(51) Int. Cl. A61M 5/172

(52) U.S. Cl. CPC A61M 5/1723

(57) ABSTRACT

The present disclosure relates to an electronic circuit for a drug delivery device. The electronic circuit including: a communication interface operable to wirelessly communicate with a mobile electronic device and to transmit at least a wireless signal to the mobile electronic device, the wireless signal allowing to determine at least one of a distance and a relative position between the electronic circuit and the mobile electronic device, and a processor operatively connected to the communication interface and being operable to enable, to disable, to trigger or to terminate execution of an electronically implemented function of the electronic circuit on the basis of an analysis of at least one of the distance and the relative position.

### CROSS REFERENCE TO RELATED APPLICATIONS

The present application is the national stage entry of International Patent Application No. PCT/EP2022/080402, filed on Nov. 1, 2022, and claims priority to Application No. EP 21315213.5, filed on Nov. 3, 2021, the disclosures of which are incorporated herein by reference.

### TECHNICAL FIELD

The present disclosure relates to an electronic circuit or unit for a drug delivery device, to a drug delivery device, to a supplementary device for a drug delivery device, to a method of controlling an electronically implemented function of or associated with a drug delivery device and to a computer program for device controlling and/or user authentication.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, e.g. injection devices and needle based injection system (NIS) devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Needle based injection to be conducted with a variety of injection devices is more and more associated with measuring and logging data regarding the amount of medicament set and set or dispensed or injected at a particular date or time. For this, injection devices may be provided with an electronic circuit providing data acquisition and/or data logging as well as communication of collected or measured dispensing related data.

Such electronic circuits or electronic units may be either implemented or integrated into the injection device itself. Alternatively, such electronic circuits or electronic units may be provided by a supplementary device or an add-on device configured for releasable attachment to the injection device and being operable to log injection-related data during use of the injection device. Moreover, electronic circuits for data logging may be also provided with a communication interface so as to transmit previously stored injection-related data to an external electronic device for further data processing.

Some users or patients may need to administer different medicaments by using different drug delivery devices, where the devices may, at first glance, look similar. Also, devices may be kept in households, where not only a single but a plurality of persons is in theory able to access the device. Moreover, some patients may have difficulties in locating, finding or retrieving a drug delivery device in their household.

### SUMMARY

The present disclosure provides an improvement to electronic circuits for drug delivery devices and provides a reliable, intuitive and smooth support for a user to use a drug delivery device. The present disclosure provides a computer program and respective hardware components to enhance patient safety, to prevent unauthorized use of a drug delivery device by a user not authorized to make use of the drug delivery device. Moreover, the present disclosure aims to enable or to simplify a process of locating a drug delivery device in case it gets lost.

In one aspect, the present disclosure relates to an electronic circuit for a drug delivery device. The drug delivery device is operable to dispense a medicament, e.g. a liquid medicament by way of injection. The electronic circuit comprises a communication interface, which is operable to wirelessly communicate with a mobile electronic device and which is also operable to transmit at least a wireless signal to the mobile electronic device. The wireless signal allows to determine and/or to quantitatively measure at least one of a distance and a relative position between the electronic circuit and the mobile electronic device. The electronic circuit further comprises a processor operationally connected to the communication interface. The processor is operable to enable, to disable, to trigger or to terminate execution of an electronically implemented function of the electronic circuit on the basis of an analysis of at least one of the distance and the relative position.

Typically, the processor is operable to approve or to disapprove access to the electronically implemented function of the electronic circuit. When the processor approves access to the electronically implemented function the processor is operable to enable, to disable, to trigger or to terminal execution of the respective function. When the processor disapproves access to the electronically implemented function, e.g. when the distance or the relative position as determined on the basis of the wireless signal is out of a predefined range, the processor is inoperable to enable, to disable, to trigger or to terminal execution of the electronically implemented function of the electronic circuit.

The electronic circuit is operable to communicate with the mobile electronic device, in particular with a counterpart communication interface of the mobile electronic device in order to quantitatively measure or to determine at least one of a distance between the electronic circuit and the mobile electronic device. Additionally or alternatively, the electronic circuit and the wireless communication with the mobile electronic device serves to determine and/or to measure a relative position of the electronic circuit and the mobile electronic device.

A relative position between the electronic circuit and the mobile electronic device may indicate the distance between these objects as well as a direction. From the perspective of e.g. the mobile electronic device a relative position is indicative about the direction and the distance where the electronic circuit is actually positioned relative to the mobile electronic device. The relative position may be characterized by a distance and by a distance vector being indicative about the relative angle or direction of the shortest distance between the electronic circuit and the mobile electronic device.

By determining at least one of a distance and a relative position between the electronic circuit and the mobile electronic device a particular function of the electronic circuit is one of enabled, disabled, e.g. triggered or terminated by using the distance and/or the relative position as a control parameter.

In some implementations, access to the electronically implemented function of the electronic circuit is provided when the distance is below a predefined approval distance. As long as the distance is above the predefined approval distance, the processor may be configured to disapprove access to the electronically implemented function of the electronic circuit. In the same way not only the distance but also a distance vector and/or an orientation of the electronic circuit relative to the mobile electronic device may be used as a control parameter.

By measuring at least one of the distance or the relative position and using this parameter as a control parameter for granting access to an electronically implemented function of the electronic circuit the mobile electronic device, the wireless communication with the mobile electronic device may provide a key lock, e.g. to unlock or to approve an electronically implemented function of the electronic circuit.

In some implementations, one of the processor, the wireless communication interface or the mobile electronic device is configured to measure and/or to determine the at least one of the distance or the relative position between the electronic circuit and the mobile electronic device. In some implementations, one of the communication interface or the processor of the electronic circuit is operable to derive and/or to measure and/or to determine at least one of the distance or the relative position between the electronic circuit and the mobile electronic device, e.g. by processing of a wireless communication signal as obtained or received by the communication interface.

In some other implementations, the mobile electronic device is configured to determine, to derive or to calculate the distance and/or the relative position between the electronic circuit and the mobile electronic device. Here, the distance and/or the relative position as determined or calculated by the mobile electronic device may be transmitted to the electronic circuit through a wireless communication link established between the counterpart communication interface of the mobile electronic device and the communication interface of the electronic circuit. Here, the electronic circuit may be inoperable to derive or to calculate the distance and/or the relative position between the electronic circuit and the mobile electronic device. Rather, respective information about at least one of the distance and the relative position may be derived by the mobile electronic device and may be thus transmitted through a wireless communication link to the communication interface of the electronic circuit.

This way, the respective distance or relative position information is made available to the electronic circuit. Here, computational power for calculating at least one of the distance and the relative position can be exclusively provided by the mobile electronic device, which is e.g. implemented as one of a smartphone, as a smartwatch, as a fitness tracker and the like wearable electronic device.

According to a further example the processor of the electronic circuit is operable to analyze at least one of the distance and the relative position. Here, the at least one of the distance and the relative position may be derived or calculated by the processor. Alternatively, the distance and/or the relative position may be obtained through wireless communication with the mobile electronic device.

In some implementations, the communication interface of the electronic circuit is implemented as a transceiver operable to transmit as well as to receive wireless signals to and/or from a counterpart communication interface of the mobile electronic device. In some implementations, the mobile electronic device may initially transfer or broadcast wireless signals to be received by the communication interface of the electronic circuit. Here, the communication interface of the electronic circuit may be configured to process a wireless signal received from the counterpart communication interface and may be further configured to generate a response wireless signal as well as to transmit a such a response wireless signal to the counterpart communication interface. Hence, the communication interface of the electronic circuit may be implemented to provide the bi-directional wireless communication with the counterpart communication interface of the mobile electronic device.

In some implementations, the communication interface is operable to modify a wireless signal received from the counterpart communication interface of the mobile electronic device and to return or to transmit the correspondingly modified wireless signal to the counterpart communication interface. The counterpart communication interface and an optional processor of the mobile electronic device may be operable to derive or to calculate at least one of the distance and the relative position from the response signal of the communication interface of the electronic circuit.

In some implementations, the communication interface of the electronic circuit may initiate transmission of wireless signals and which listens to returning wireless signals of at least one mobile electronic device. Here, the counterpart communication interface of the mobile electronic device may be configured to receive, to process and/or to modify wireless signals initiated and/or transmitted by the communication interface of the electronic circuit. In either way, the communication interface of the electronic circuit as well as the counterpart communication interface of the mobile electronic device are implemented as a transceiver being operable to transmit and to receive wireless signals.

According to a further example the electronic circuit comprises a proximity sensor operable to measure the at least one of the distance and the relative position between the electronic circuit and the at least one of the mobile electronic device. The proximity sensor may be implemented by a wireless communication between the communication interface of the electronic circuit with a counterpart communication interface of the at least one mobile electronic device. The proximity sensor may be implemented in the communication interface. In some other implementations, the communication interface constitutes or forms the proximity sensor.

In effect and by measuring at least one of a distance and a relative position between the electronic circuit integrate into or attached to the drug delivery device and the at least one mobile electronic device respective electronically implemented function(s) of the electronic circuit and/or of the drug delivery device may be conducted or executed in dependency of one of a distance and a relative position between the electronic circuit and the at least one mobile electronic device. In this way it can be effectively guaranteed, that an electronically implemented device function is only and/or exclusively enabled, disabled, triggered or terminated when and the mobile electronic device is within a certain geometric range or direction to the electronic circuit.

According to a further example the proximity sensor is operably connected to the processor. Here, the processor may be operable to evaluate the at least one identification signal only when the distance between the electronic circuit and the at least one mobile electronic device is within a predefined operating distance and/or when the electronic circuit is within a predefined relative operating position with regard to the at least one mobile electronic device.

By way of the proximity sensor there may be provided a rather automated unlocking of a particular function of the drug delivery device. In some implementations, wherein the drug delivery device is equipped with an electromechanical interlock that is controllable by the electronically implemented function of the electronic circuit, approaching of the mobile electronic device to the electronic circuit and hence to the proximity sensor thereof may trigger a release or deactivation of the interlock when the identification signal transmitted from the mobile electronic device matches with the user specific data record.

According to a further example at least one of the communication interface and the proximity sensor is implemented as a UWB (ultra-wideband) based proximity sensor. The proximity sensor is configured to quantitatively measure the at least one of the distance and the relative position between the electronic circuit and the at least one mobile electronic device on the basis of transceiving of electromagnetic ultra-wideband signals.

Typically, electromagnetic UWB signals include very short RF pulses covering a comparatively large portion of the radio spectrum at a comparatively low energy level. By way of an UWB-based communication link between the proximity sensor and the at least one mobile electronic device the distance between the electronic circuit and the mobile electronic device can be determined with high precision, e.g. in the range of a few centimeters or even below.

By way of a UWB-based communication between the proximity sensor and hence between the electronic circuit and the at least one mobile electronic device there may be also provided a relative position information being indicative of a relative position and/or orientation of the electronic circuit in relation to the at least one mobile electronic device; and vice versa.

UWB-based wireless communication enables to obtain not only a distance an object but also to measure or to determine a direction, where an object is actually positioned or located in relation to another object. The UWB-based communication between the proximity sensor and the at least one mobile electronic device is of particular use to e.g. implement a pager function with the mobile electronic device, thus allowing to find the electronic circuit attached to or integrated into the drug delivery device.

With a further implementation of the electronic circuit, the processor is operable to generate a control command on the basis of the analysis of the at least one of the distance and the relative position between the electronic circuit and the mobile electronic device. The control command is configured to cause at least one of:
generating a user-perceivable alert or signal by a signaling unit,
locking or unlocking a drive mechanism of the drug delivery device by an electromechanical interlock,
triggering or terminating transmission of data by the communication interface,
switching the electronic circuit from a standby mode into an active mode, or vice versa, switching of the electronic circuit from an active mode into a standby mode.

In some implementations the processor generates a control command causing the signaling unit to generate one of an approval or confirmation signal, e.g. when the distance between the electronic circuit and the mobile electronic device is within a predefined approved or approval distance, respectively. The user-perceivable signal or alert may indicate to a user, that e.g. a function of the electronic circuit and/or of the drug delivery device is currently enabled or unlocked.

In other situations and e.g. when the distance is above the approval distance the processor may be operable to generate a control command that causes the signaling unit to generate a user-perceivable alert or signal that indicates a locking of the respective electronically implemented function of the electronic circuit and/or of the drug delivery device. In some implementations, and as long as the distance between the mobile electronic device and the electronic circuit is above the predefined approval distance the drive mechanism of the drug delivery device may be mechanically locked or blocked. Additionally or concurrently, the signaling unit may be operable to generate a respective indication to the user.

According to a further example the communication interface of the electronic circuit is configured to exchange user specific data with the mobile electronic device. The user specific data includes at least one of:
a medicament type,
a time and/or a date of a medicament dispensing action,
an amount of a medicament dispensed,
motion data of the user.

The wireless communication link as established between the communication interface of the electronic circuit and the counterpart communication interface of the mobile electronic device is not only operable to exchange wireless signals for measuring or determining at least one of the distance and the relative position but may be also used to exchange user specific data or further data being specific about the configuration of the drug delivery device. Insofar, the wireless communication link between the electronic circuit and the mobile electronic device provides a twofold or multi-purpose function, namely to measure a distance and to transfer user-specific and/or device-specific information.

In some implementations, the electronic circuit is implemented in an electronic unit of a drug delivery device. The drug delivery device may be implemented as an injection device, e.g. as a pen-type injector. In some other implementations, the electronic circuit is part of or is integrated into a supplementary device configured for attachment or fastening and/or fixing to a housing of an injection device.

According to another aspect the present disclosure further relates to a drug delivery device operable to dispense a medicament, e.g. operable to dispense a medicament by way of injection. The drug delivery device comprises a housing to accommodate a medicament container filled with the medicament. Typically, the medicament container comprises a cartridge filled with a liquid medicament configured for injection into biological tissue of a patient. The drug delivery device further comprises a drive mechanism operatively connectable to the medicament container in order to expel or to withdraw a dose of the medicament from the medicament container. The drug delivery device further comprises an electronic circuit as described above integrated into the housing and/or operatively connected to the drive mechanism.

When implemented in or on the drug delivery device the electronic circuit may be also operable to monitor operation of the drug delivery device. The electronic circuit may be operable to measure and/or to record setting and/or dispensing of a dose of the medicament as initiated or conducted by a user of the drug delivery device.

Since the drug delivery device comprises an electronic circuit as described above, all features, effects and benefits as described above in connection with the electronic circuit for a drug delivery device equally apply to the drug delivery device; and vice versa.

According to another aspect the present disclosure relates to a supplementary device or add-on device for a drug delivery device as described above. The supplementary device comprises a housing and a fastener for fastening and/or fixing the supplementary device to a housing of the drug delivery device. Furthermore, the supplementary device comprises an electronic circuit as described above. Insofar all features, effects and benefits as described above in connection with the electronic circuit equally apply to the supplementary device; and vice versa.

The supplementary device may be further operable to measure or to determine a configuration of the drug delivery device when attached to the drug delivery device. The supplementary device may be particularly configured to measure a size of a dose currently set or dispensed by the injection device. Typically, the supplementary device is operable and/or configured to monitor repeated or frequent use of the drug delivery device.

The supplementary device may be configured to generate and/or to store as well as to communicate a dosing or injection history as conducted by the injection device. Typically, the supplementary device is operable to store and/or to monitor a date and a time at which an amount of medicament has been dispensed or injected by the drug delivery device. The particular amount of the medicament, e.g. a dose of constant or variable size may be determined, measured and/or recorded as well.

In another aspect the present disclosure relates to a mobile electronic device for use with a drug delivery device. The mobile electronic device comprises a mobile communication interface operable to wirelessly communicate with a mobile or counterpart communication interface of an electronic circuit associated with the drug delivery device. The mobile communication interface is typically operable to transmit at least a wireless signal to the communication interface. The wireless signal(s) allow(s) to determine at least one of a distance and a relative position between the electronic circuit and the mobile electronic device.

The mobile electronic device further comprises a processor operatively connected to the mobile communication interface. The processor is operable to enable, to disable, to trigger or to terminate execution of an electronically implemented function of the mobile electronic device on the basis of an analysis of at least one of the distance and the relative position.

Here and concurrently with the above-mentioned electronic circuit or the drug delivery device it is the processor of the mobile electronic device that is operable to process at least one of the distance and the relative position between the electronic circuit and the mobile electronic device in order to provide or to deny, hence to approve or to disapprove access to the electronically implemented function of the mobile electronic device.

Hence, a particular function of the mobile electronic device, e.g. a function of the mobile electronic device to cooperate with the drug delivery device may be disabled as long as the distance and/or the relative position between the mobile electronic device and the electronic circuit is beyond or outside a predefined approval range, distance or position.

In some implementations, the electronically implemented function of the mobile electronic device comprises readout of device-specific or, medicament-specific or user-specific data of the electronic circuit associated with the drug delivery device. Hence, a readout of data and/or establishing of a respective data transferring communication link requires that the distance between the mobile electronic device and the electronic circuit is within a predefined approval distance.

In some implementations, and when the mobile electronic device may be implemented as a remote control or remote controller of the drug delivery device it may be required to reduce the distance between the drug delivery device associated or provided with the electronic circuit and the mobile electronic device to lie within the approved distance. As long as the mobile electronic device is located outside a predefined approval distance or approval position a remote controlling function of the mobile electronic device may be disabled. When the distance between the mobile electronic device and the electronic circuit is within a predefined approval distance and/or when the relative position of the mobile electronic device relative to the electronic circuit is within a predefined approved relative positional range a respective function of the mobile electronic device may be enabled.

This way, a kind of a remote control for and/or a particular drug delivery device related function of the mobile electronic device requires to bring the mobile electronic device in close vicinity to the drug delivery device. By way of this requirement patient safety and compliance with an intended use of the drug delivery device can be enhanced.

In some other implementations, the mobile electronic device comprises a proximity sensor operatively connected to the processor and operable to measure at least one of the distance and the relative position between the electronic circuit and the mobile electronic device. The proximity sensor may be integrated into the mobile communication interface. In some implementations, the mobile communication interface provides or contributes to the proximity sensor.

The mobile communication interface may provide or may coincide with the counterpart communication interface as described above in connection with the mobile electronic device. It is hence operable and configured to transmit electromagnetic signals wirelessly to the communication interface of the electronic circuit. It is also configured to receive respective wireless signals, e.g. response signals from the communication interface of the electronic circuit, e.g. in return.

In some other implementations, at least one of the mobile communication interface and the proximity sensor comprises a UWB antenna operable to transmit and/or to receive ultra-wideband signals. The UWB antenna is typically integrated in one of the proximity sensor and the communication interface as described above.

In some other implementations, the mobile electronic device further comprises a signaling unit, which is operable to indicate and/or to illustrate to a user at least one of:
the distance between the electronic circuit and the mobile electronic device, and
the relative position of the electronic circuit in relation to a position of the mobile electronic device.

The signaling unit may comprise a one or two-dimensional display configured to visually illustrate at least one of the distance and the relative position. In some other implementations, the signaling unit may simply comprise a light source operable to generate and to provide a visible light signal. The signaling unit may be operable to provide light signals of different color, different brightness and/or of different duration. Hence, at least one of a distance and a relative position between the electronic circuit and the mobile electronic device may be indicated by the light source, e.g. by varying a least one, some or all of a frequency, brightness and duration of a blinking light, by varying an intensity of a light signal and/or by varying a color of the light signal.

In some other implementations, the signaling unit comprises a speaker or the like hardware component to generate an acoustic signal. Also here, the strength or intensity as well as duration and frequency of the acoustic signal may be configured to indicate at least one of the distance and the relative position.

In some other implementations, the signaling unit is operable to generate a haptically perceivable signal, e.g. by generating a mechanical vibration or by generating a mechanically or haptically perceivable knocking signal. Also here and by varying at least one of an intensity, a duration and a frequency of the haptic signal the signaling unit may be indicative of at least one of a distance and a relative position between the electronic circuit and the mobile electronic device.

According to a further example the mobile electronic device is implemented as an external electronic device, being separated from the electronic circuit of the drug delivery device or being separated from the electronic circuit of a supplementary device attachable and/or fixable to the drug delivery device. In some implementations, the mobile electronic device is a wearable electronic device. In some implementations, the mobile electronic device is implemented as a smartphone, as a tablet computer, as a smartwatch, as a fitness tracker or as a wireless tag.

In some implementations, the mobile electronic device is implemented as a mobile electronic device to communicate with an electronic circuit as described above. Insofar, all features, effects and benefits described above in connection with the electronic circuit, with the drug delivery device, and/or with the supplementary device may equally or concurrently apply to the mobile electronic device; and vice versa.

According to another aspect, the disclosure also relates to a method of controlling an electronically implemented function associated with a drug delivery device, e.g. with a drug delivery device as described above. The method comprises the steps of establishing of a wireless communication link between an electronic circuit attached to or integrated into the drug delivery device and a mobile electronic device. The method further comprises a step of determining at least one of a distance and a relative position between the electronic circuit and the mobile electronic device through transmission of wireless signals between the electronic circuit and the mobile electronic device. The method further comprises a step of analyzing or evaluating at least one of the distance and the relative position. Furthermore, the method comprises at least one of enabling, disabling, triggering or terminating execution of an electronically implemented function of at least one of the electronic circuit and the mobile electronic device on the basis of the analysis or evaluation of at least one of the distance and the relative position.

In some implementations, the method may be implemented by at least one or several of an electronic circuit as described above, a drug delivery device as described above, a supplementary device as described above and a mobile electronic device as described above.

In some implementations, the method is conducted or executed through wireless communication between the electronic circuit, typically implemented in one of the drug delivery device and the supplementary device and a mobile electronic device as described above.

In some implementations, the at least one of enabling, disabling, triggering or terminating execution of an electronically implemented function of at least one of the electronic circuit and the mobile electronic device includes at least one of the following:
generating a user-perceivable alert or signal by a signaling unit,
locking or unlocking a drive mechanism of the drug delivery device by an electromechanical interlock,
triggering or terminating transmission of data by the communication interface, or
switching the electronic circuit from a standby mode into an active mode, or vice versa, switching of the electronic circuit from an active mode into a standby mode.

In some implementations, the method may be also implemented by a first electronic circuit implemented in a drug delivery device, by a second electronic circuit implemented in a supplementary device and by a mobile electronic device as described above. Here, the first and the second electronic circuits each comprise an electronic circuit as described above.

According to another as a present disclosure relates to a computer program comprising computer readable instructions, which, when executed by at least one processor of an electronic circuit as described above or when executed by at least one processor of a mobile electronic device as described above cause the respective processor(s) to establish a wireless communication link between the electronic circuit attached to or integrated into the drug delivery device and the mobile electronic device of a user.

The computer readable instructions may further cause the processor to determine at least one of a distance and a relative position between the electronic circuit and the mobile electronic device through transmission of wireless signals between the electronic circuit and the mobile electronic device. The computer readable instructions may also trigger transmission and receiving of respective wireless electromagnetic signals. The computer readable instructions are further operable to cause the processor to analyze or to evaluate at least one of the distance and the relative position and to enable, to disable, to trigger or to terminate execution of an electronically implemented function of at least one of the electronic circuit and the mobile electronic device on the basis of the analysis or evaluation of at least one of the distance and the relative position.

Typically, the computer program is configured to execute the above-described method of controlling an electronically implemented function of or associated with a drug delivery device. Insofar, all features, effects and benefits as described above in connection with the electronic circuit for a drug delivery device, a drug delivery device operable to dispense a medicament, a supplementary device for such a drug delivery device the mobile electronic device for use with the drug delivery device and/or as described in connection with the method of controlling the electronically implemented function equally apply to the computer program; and vice versa.

According to a further aspect the present disclosure relates to a set comprising one of, an arbitrarily selected plurality of, or all of:
a drug delivery device provided with an electronic circuit as described above,
a supplementary device attachable to the drug delivery device and provided with an electronic circuit as described above, and
a mobile electronic device as described above. Insofar all features, effects and benefits as described above in relation to any one of the electronic circuit, the drug delivery device, the supplementary device and the mobile electronic device equally apply to this set; and vice versa.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

According to a further example the at least one wireless signal transmitted between the electronic circuit and the mobile electronic device includes acquired motion data of the user of the mobile electronic device. The processor and/or the mobile electronic device is further operable to evaluate the acquired motion data by comparing the acquired motion data with stored motion data of the user specific data record. The user specific data record may be provided in a storage of one of the electronic circuit and the mobile electronic device.

According to a further example the processor of the electronic circuit and/or a processor of the mobile electronic device is operable to generate a data matching index being indicative of a degree of matching between the acquired motion data and/or stored motion data. The processor may be further operable to enable, to disable, to trigger or to terminate execution of the electronically implemented function of the electronic circuit on the basis of the data matching index. This way, a user authorized to use the drug delivery device can be identified by motion data.

The motion data may be acquired or collected either by the electronic circuit or by the mobile electronic device, e.g. carried along with the user or attached to the user. Typically, motion data of the user is acquired over a predefined period of time and/or motion data is acquired at specific and predefined points of time or at particular days. The motion data may be characteristic and unequivocal for each user using the drug delivery device and/or using the mobile electronic device. It may provide a kind of a fingerprint of each user.

Typically, a first user is characterized by motion data of a first user specific data record. Accordingly, a second user can be characterized by motion data of a second user specific data record. By comparing motion data that has been acquired while a user being in motion or while a user executes or conducts a particular movement or motion pattern and by comparing the acquired motion data of this user with motion data of a user specific data record a user of a mobile electronic device can be identified.

Typically, and in some implementations, the user specific data record is assigned or mapped to a particular drug delivery device. When the same user makes use of the drug delivery device and when the motion data of this particular user is acquired a comparison of the acquired motion data with the user specific data record assigned or mapped to the drug delivery device shows a high degree of matching or similarity. Accordingly, the respective user will be authorized to use the drug delivery device.

Accordingly, at least one function of the electronic circuit and hence of the drug delivery device is enabled, disabled, triggered or terminated as demanded by the user of the drug delivery device. In other situations, and when a second user, which is not authorized to use the drug delivery device, intends to use this particular device, the motion data of this second user is collected or acquired and is compared with motion data of the user specific data record of the first user, which is mapped to this particular drug delivery device. Since motion data of the second user distinguishes from the motion data of the user specific data record there will be determined a rather low degree of matching or similarity between the acquired motion data of the second user and the stored motion data of the user specific data record of the first user. Accordingly, the respective function of the drug delivery device will be enabled, disabled, triggered or terminated on the basis of the data matching index.

In some implementations, and when a user is authenticated to use the drug delivery device the respective device function may be enabled or triggered. In other situations, and when analysis or evaluation of the motion data of the user reveals that the user is not authenticated to use the drug delivery device, e.g. due to a rather low degree of matching between the acquired motion data and the stored motion data, the respective function of the drug delivery device may be disabled or terminated.

According to a further example the motion data of the user is acquired by a sensor. Hence, the electronic circuit and/or the mobile electronic device comprises a sensor operatively connected to the processor. The sensor may be operable to recognize a movement or movement pattern and/or gestures of a user when moving himself or when moving at least one of a mobile electronic device and the drug delivery device. This way, a movement profile or pattern of a user as such or typical gestures of a user can be detected and/or quantitatively measured. Such movement patterns or gestures can be permanently or occasionally acquired and can be compared with movement or motion patterns and/or gesture data of the user specific data record.

In some implementations, the sensor comprises at least one of an acceleration sensor, a rotation sensor, a position sensor, a distance sensor. The sensor is operable to generate electrical sensor signals. The processor is operable to evaluate and/or to analyze the sensor signal so as to derive and/or to characterize motion data being indicative of characteristic motions or gestures of the user when moving the electronic circuit and/or the mobile electronic device and hence the sensor.

Deviations of acquired motion data from stored motion data can be detected, thus leading to a rather low degree of matching and to the generation of a rather low matching index. Depending on the size of the matching index execution of the function of the electronic circuit and/or of the associated drug delivery device, which typically requires a respective authorization can be enabled, disabled, triggered or terminated.

When the sensor comprises a position sensor it may be operable to determine an absolute or relative position, e.g. by way of communicating with a satellite-based positioning system or by communicating with another electronic device. This way, either an absolute position of the sensor, e.g. in form of geographic coordinates or e.g. in form of relative coordinates in relation to another electronic device can be measured or determined.

In some other implementations, the electronic circuit is also equipped with a clock by way of which the motion data or position data acquired by the sensor can be mapped to a date, to a point of time and/or to a time interval. This way, a motion profile or movement profile over time can be generated being indicative of a typical motion pattern of a user.

As an example, a user may be identified to be at a certain geographic position at a typical point of time or time interval. For instance, a first user may be characterized by staying 8 hours a day, e.g. from 9 to 17 o'clock in a particular office building. A user may leave the office building for a lunch break at a regular time and for a time interval of e.g. half an hour.

Moreover, a user may be recognized by moving from home to work e.g. by car or by public transportation at a particular time of the day. Such motion data may be captured and acquired over a comparatively long time interval. When motion data currently acquired should strongly deviate from the long-term average of respective motion data this may be indicative of a situation, in which a different user uses the respective electronic circuit.

Deviations of acquired motion data from stored motion data or average motion data may be quantitatively expressed by the data matching index. This way, there can be generated a probability or a likelihood if and in how far motion data currently acquired is obtained from a particular user mapped or assigned to the user specific data record.

According to a further example at least a portion of the motion data of the user is acquired by the communication interface through wireless communication with at least one of a satellite-based positioning system, an access point of a communication network, a counterpart communication interface of a mobile electronic device, such as a supplementary device attachable to the drug delivery device, an electronic unit or electronic circuit of the drug delivery device or a mobile electronic device.

This way, there is provided a high degree of flexibility of how to obtain motion data of a user. Insofar, motion data of a user can be obtained even by way of an electronic device being void of a sensor, such as an acceleration sensor, a rotation sensor, a position sensor and a distance sensor. For instance in an urban area there may be provided numerous access points of a communication network to which the communication interface of the electronic circuit establishes a communication link as the electronic circuit is subject to a movement or motion. Simply by logging establishing of communication links with different access points of a communication network during a movement or motion from one place to another there can be generated a motion pattern or motion profile which may be indicative of a habitual movement or motion of the user.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F (ab′) 2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a method of controlling an electronically implemented function of or associated with a drug delivery device as well as numerous hardware configurations including an authentication system, an electronic circuit, a drug delivery device, a supplementary device and numerous implementations of mobile electronic devices will be described in greater detail by making reference to the drawings, in which:

FIG. 1 illustrates an example of a drug delivery device implemented as a pen-type injection device,

FIG. 2 shows an injection device equipped with a supplementary device,

FIG. 3 shows a longitudinal cross-section through an example of a pen-type injection device,

FIG. 4 shows numerous hardware components of an authentication system,

FIG. 5 is illustrative of a block diagram of an example of the authentication system,

FIG. 6 is illustrative of a block diagram of another implementation of the authentication system,

FIG. 7 shows a block diagram of a further implementation of the authentication system,

FIG. 8 is illustrative of a block diagram, wherein a supplementary device attached to an injection device is operable to communicate with at least one or a plurality of mobile electronic devices,

FIG. 9 is illustrative of a wireless communication between a mobile electronic device and an electronic unit of the drug delivery device,

FIG. 10 schematically illustrates different users provided assigned with different data records,

FIG. 11 shows a flowchart of a method to authenticate a user to use a drug delivery device,

FIG. 12 is indicative of a comparison of acquired motion data with stored motion data,

FIG. 13 shows a flowchart of a further implementation of the method of authenticating a user, and

FIG. 14 shows a further flowchart of the method of authenticating the user.

### DETAILED DESCRIPTION

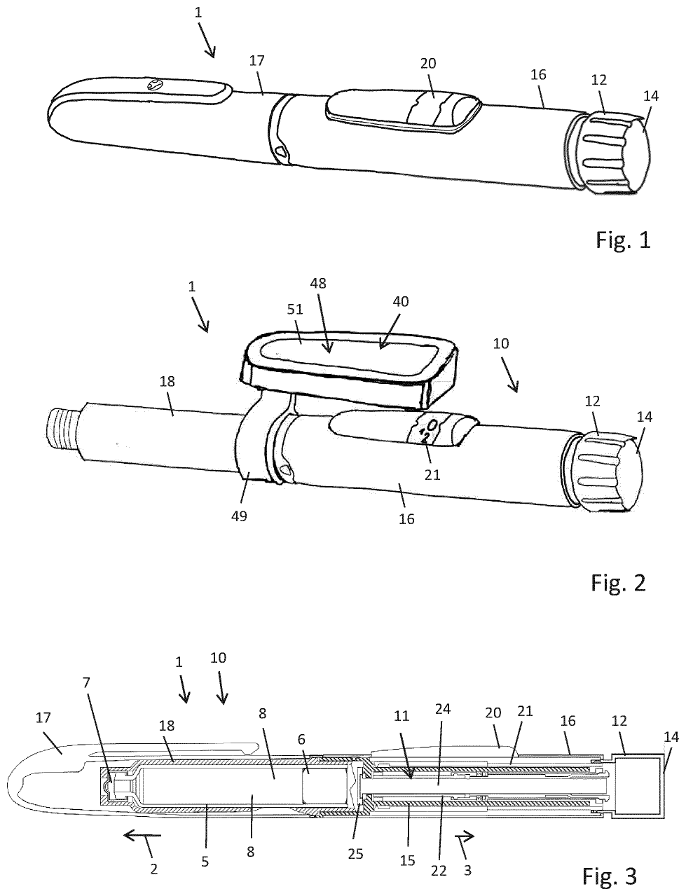

In FIGS. 1-3 an example of a drug delivery device 1 implemented as an injection device in is schematically illustrated. The drug delivery device 1 comprises an elongated housing 16, e.g. of cylindrical or tubular shape. The housing 16 extends along a longitudinal direction. Towards a distal direction 2 and hence towards a distal end an injection needle 19 can be attached to a container housing 18 forming or constituting a distal part of the housing of the injection device 10. Towards a proximal direction 3 and hence at a proximal end of the injection device 10 there is provided a dose dial 12 and/or a dose button 14 allowing to set a dose and to dispense a dose of the medicament 8, respectively.

The container housing 18 is configured and shaped to accommodate a medicament container 5. The medicament container 5 may comprise a tubular-shaped barrel. The medicament container 5, e.g. implemented as a standard cartridge may be sealed towards the proximal direction 3 by way of a movable stopper 6. The distal end of the medicament container 5 is typically sealed by a pierceable seal 7. Hence, an outlet of the medicament container 5 is covered by the pierceable seal and is fixed to a head of the medicament container 5 e.g. by a crimped cap (not illustrated).

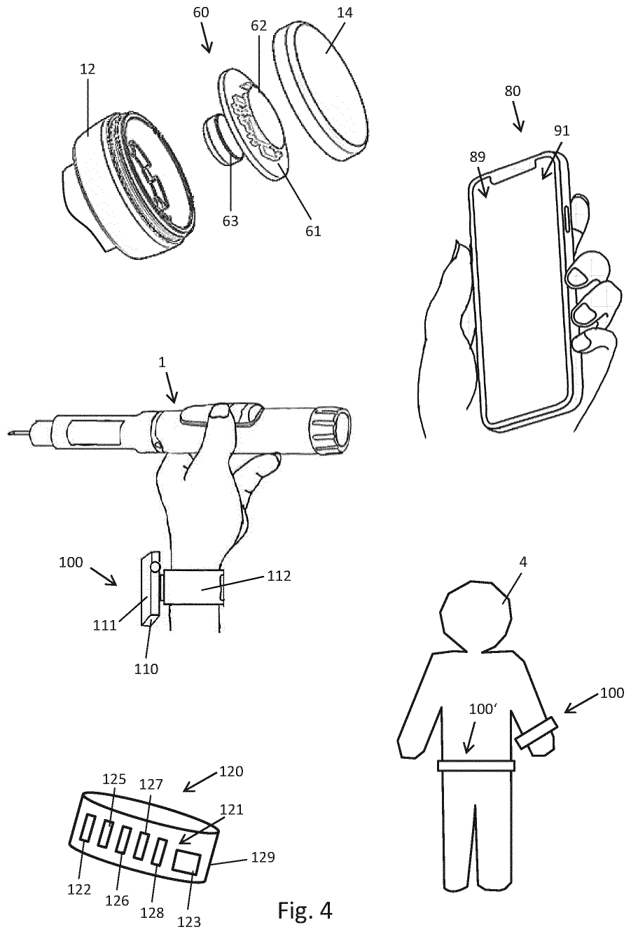

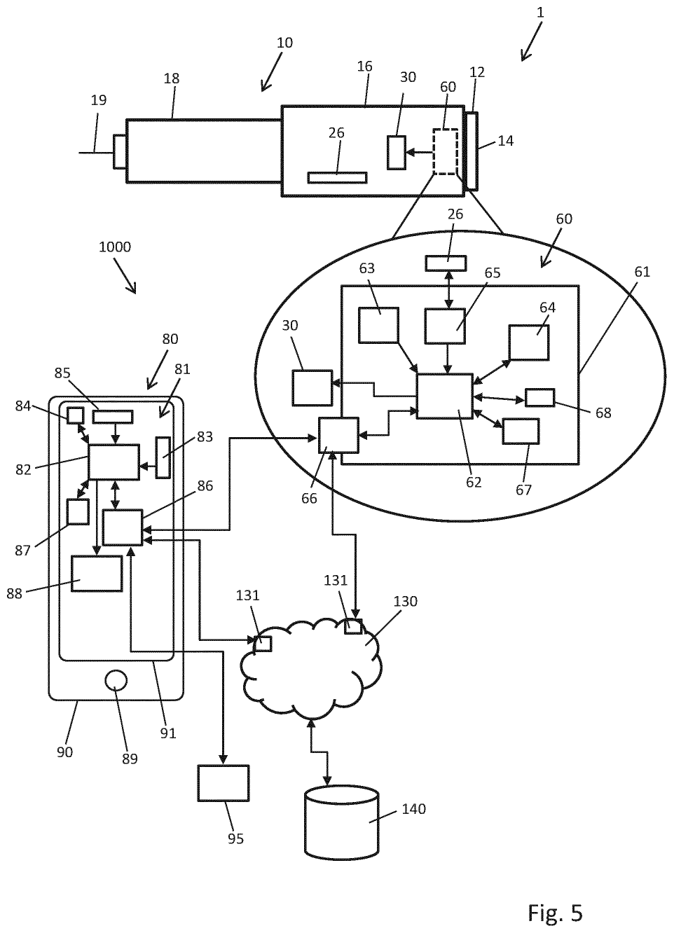

The injection device 10 further comprises a drive mechanism in 11. The drive mechanism 11 comprises a piston rod 24 of elongated shape and extending along the longitudinal direction of the housing 16. Inside the housing 16 there may be provided an inner body 15 serving as a support or mount of the drive mechanism 11. The piston rod 24 may be threadedly engaged with the inner body 15. A rotation of the piston rod 24 as induced by the drive mechanism 11 may thus lead to an advancing motion of the piston rod 24 relative to the inner body 15 and relative to the housing 16 so as to urge or to move the stopper 6 in distal direction 2 relative to the medicament container 5. This way, a dose of the medicament 8 can be expelled from the outlet of the medicament container 5. Typically, the seal 7 is pierceable by a double-tipped injection needle 19 as illustrated in FIGS. 4 and 5. The injection needle, e.g. comprising a threaded needle hub may be detachably or releasably connected to a distal end of the container housing.

The drive mechanism 11 typically comprises a number sleeve 21 at least partially visible through a window 20 provided in the housing 16 of the injection device 10. Upon setting a dose the number sleeve 21 is subject to a rotating motion. Accordingly, an increasing sequence of dose indicating numbers may show up in the window 20 indicating the size of a dose currently set. The drive mechanism 11 further comprises a drive sleeve 22. The drive sleeve 22 is operatively connected or coupled with the piston rod when the drive mechanism 11 is in a dose dispensing or dose injection mode. Pushing the dose button 14 may initiate a rotation of the drive sleeve 22, which by activation of a clutch is operatively connected with the piston rod 24 so as to induce a dose dispensing rotation and a distally directed longitudinal advancing motion of the piston rod 24.

The piston rod 24 is provided with a pressure please 25 at its distal end. The pressure piece 25 is typically rotationally supported at the distal end of the piston rod 24. By way of the pressure piece 25 distally directed thrust as exerted by the piston rod 24 is transferred onto a proximal thrust receiving surface of the stopper 6. In some implementations, the drive mechanism 11 is provided with a mechanical energy storage, such as a spring. The mechanical energy storage may be biased during or upon setting of a dose. By depressing the dose button 14 mechanical energy stored in the mechanical energy storage may be released so as to provide a driving torque or driving force capable to advance the piston rod 24 in distal direction 2 for dispensing of a dose of the medicament 8. With other examples the drive mechanism 11 is void of a mechanical energy storage. Here, a force exerted by the user onto the dose button 14 is nearly entirely transferred into a driving force required for moving the piston rod 24 in distal direction.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1: 2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

The drive mechanism 11 as described above is only exemplary for one of a plurality of differently configured drive mechanisms that are generally implementable in a disposable or reusable pen-injector. The drive mechanism as described above is explained in more detail e.g. in WO2004/078239A1, WO 2004/078240A1 or WO 2004/078241A1 the entirety of which being incorporated herein by reference. Further examples of drive mechanisms 11 to be implemented with the injection device 10 can be found in WO 2014/033195 A1 or WO 2014/033197 A1 the entirety of which being incorporated herein by reference. The drive mechanism as disclosed in WO 2014/033195 A1 is a reusable drive mechanism. The drive mechanism as disclosed in WO 2014/033197 A1 is an example of a disposable drive mechanism being void of a reset function.

In FIG. 4 numerous components of an authentication system 1000 as described below are schematically illustrated. The injection device 10 may be equipped with an electronic unit 60. The electronic unit 60 typically comprises an electronic circuit 61. The electronic circuit 61 comprises a processor 62 and a battery 63. Moreover and with typical examples, the electronic unit 60 embedded into or integrated into the injection device 10 may also comprise a storage 64, a sensor 65, a communication interface 66, a proximity sensor 67, a signaling unit 68.

As indicated in FIG. 4, the electronic unit 60 may be arranged inside the dose dial 12. The dose button 14 may close a receptacle of the dose dial 12, which receptacle is sized to accommodate at least a portion of the electronic unit 60. As further illustrated in FIG. 4 the authentication system 1000 may comprise at least one mobile electronic device 80, 100, 120. The mobile electronic device 80 may be implemented as an external electronic device. It may comprise a smartphone. The smartphone 80 typically comprises an input 89 and a display 91. Here, the input 89 may be integrated into the display 91, which is hence implemented as a touch sensitive display. With other examples the mobile electronic device 100 is implemented as a wearable electronic device. The wearable electronic device 100 may comprise a strap 112 or a wristband allowing to attach the mobile electronic device 100 e.g. to a wrist of a hand of a user. The mobile electronic device 100 may be implemented as a smartwatch.

Typically, the mobile electronic device comprises a housing 110 accommodating an electronic circuit 101 equipped with a processor 102, a battery 103, a storage 104, a sensor 105, a communication interface 106, a proximity sensor 107 and a signaling unit 108. The housing 110 may be closed or covered by a display 111. In some implementations, the mobile electronic device 100 is attachable to a wrist of a hand or arm of a user 4. With other examples the mobile electronic device 100′ may be attached to another portion of a body of a user. As an example, the mobile electronic device 100′ may be implemented as a belt or may be integrated into a belt of a user.

The mobile electronic device 120 may be implemented as a wireless tag. The wireless tank 120 may comprise a housing 129. Typically, the wireless tag 120 comprises an electronic circuit 121, e.g. comprising a printed circuit board. The electronic circuit 121 may comprise at least one of a processor 122, a battery 123, a sensor 125, a communication interface 126, a proximity sensor 127 and a signaling unit 128. The mobile electronic device 120 is typically carried along by the user 4.

In some implementations, the mobile electronic device 120 may be permanently attached to a personal item of a user, such as a key. With further examples the mobile electronic device 120 may be attached to the drug delivery device 1.

With the example of FIG. 5, the drug delivery device 1 and hence the injection device 10 is further equipped with a movable part 26. The movable part 26 may be implemented in the drive mechanism 11. A movable component of the drive mechanism 11 may form or constitute the movable part 26. A movement or position of the movable parts 26 of the drive mechanism 11 may be detected or tracked by the electronic unit 60 of the injection device 10.

A detectable position or orientation of the movable part 26 may be indicative of a current state or configuration of the injection device 10. A position of the movable part 26 along the longitudinal direction may be directly indicative of a residual amount of medicament 8 provided in the medicament container 5. By detecting or quantitatively measuring at least one of a position or orientation of the movable part 26, the electronic unit 60 may gather device specific information about the momentary status or configuration of the injection device. This way, the electronic unit 60 may be configured to collect and/or to gather data being indicative of an operational status or history of use of the injection device 10. The electronic unit 60 may collect data such as a dosing or dispensing history including information about an amount of medicament being dispensed at a particular date and/or time.

The injection device 10 may be further equipped with an electromechanical interlock 30. The electromechanical interlock 30 may be integrated into the drive mechanism 11. The interlock 30 may be operable by the electronic unit 60 of the injection device 10.

With the example of FIG. 5 the injection device 10 is equipped with the electronic unit 60. The electronic unit 60 comprises e.g. a printed electronic circuit 61, a processor 62, a battery 63, a storage 64, at least one sensor 65, a communication interface 66, a proximity sensor 67 and/or a signaling unit 68.

The signaling unit 68 is configured to generate a user perceivable signal. It may comprise a speaker to generate an audible signal. It may comprise a display to visualize information to a user. It may comprise a blinking light, such as a LED. It may comprise numerous LEDs or light sources configured to generate light indication or light pulses of different color and/or duration. The signaling unit 68 may also comprise a buzzer or the like electromechanical unit to generate a vibration or some other haptically detectable signal.

The communication interface 66 is configured to communicate with another communication interface 86 of a mobile electronic device 80. Alternatively or additionally, the communication interface 66 is configured to communicate with a database 140 via a network 130. The communication interface 66 may access the database 140 and/or the network 130 by a particular access point 131. The communication between the communication interface 66 and the network 130 is typically implemented as a wireless communication link.

The mobile electronic device 80 may be implemented as a smartphone. It comprises an electronic circuit 81, a processor 82, a battery 83, a storage 84, a sensor 85, a communication interface 86, a proximity sensor 87, a signaling unit 88, a user operable input 89, a housing 90 and a display 91. The mobile electronic device 80 may be configured to communicate with a satellite-based positioning system 95 so as to gather global or regional position information. Typically, the communication interface 86 of the mobile electronic device 80 is configured to communicate with the network 130 by at least one of several access points. It may communicate with the database 140 to gather or to receive requested data from the database 140.

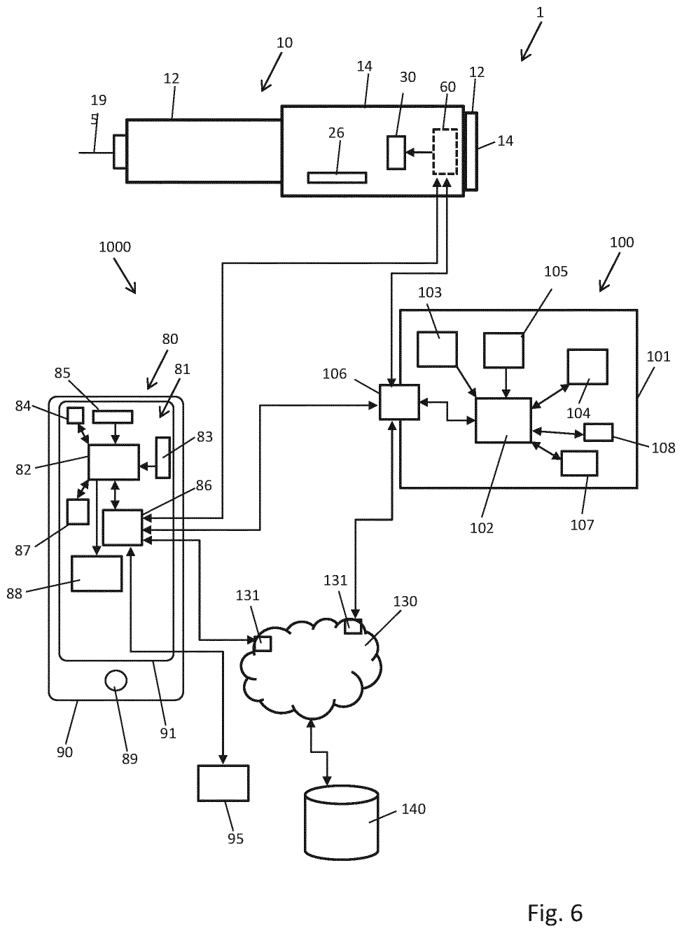

With the further example of an authentication system 1000 as illustrated in FIG. 6 there is provided not only one mobile electronic device 80 but also a further mobile electronic device 100, e.g. in form of a smartwatch or a fitness tracker. The mobile electronic device 100 also comprises at least one of an electronic circuit 101, a processor 102, a battery 103, a storage 104, a sensor 105, a communication interface 106, a proximity sensor 107, and a signaling unit 108. These components may be all encapsulated in a housing 110. The signaling unit 108 may comprise a display 111. The signaling unit 108 may be implemented in the same or a similar way as the signaling unit 68 as described above in connection with the electronic unit 60.

The mobile electronic device 100 is operable to communicate with the network 130 and the database 140 via the communication interface 106. The mobile electronic device 100 may also wirelessly communicate with the electronic unit 60 of the drug delivery device 1 as well as with the communication interface 86 of the mobile electronic device 80. With further examples the mobile electronic device 100 may also directly or indirectly communicate with the satellite-based positioning system 95 so as to obtain geographic position data, e.g. for acquiring absolute or relative position data of the mobile electronic device 100.

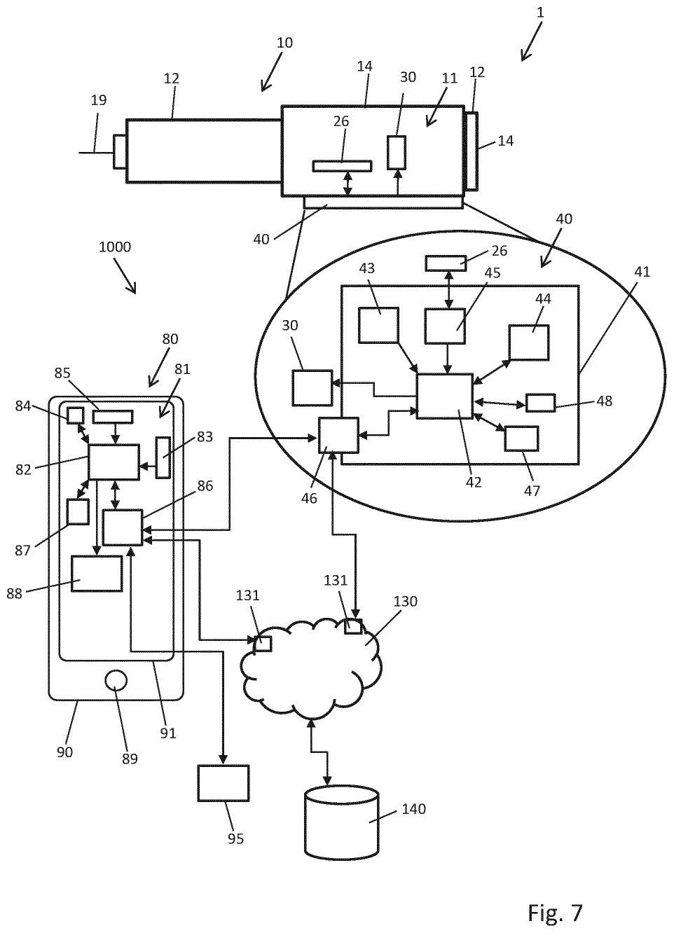

With the further example as illustrated in FIG. 7 the drug delivery device 1 may be void of an electronic unit 60 as described above. The injection device 10 and/or the drive mechanism 11 may be implemented all mechanically. Here, the injection device 10 may be equipped with a supplementary device 40. The supplementary device 40 typically comprises a fastener 49 for detachably fastening or fixing of the supplementary device 40 to the housing 16 of the injection device 10.

The supplementary device 40 comprises a signaling unit 48. The signaling unit 48 may comprise one of a light source, e.g. in form of a blinking light, a display, a speaker and a vibration generating unit. When implemented as a display or blinking light it may provide visible information or an visible alert to a user. Typically, the supplementary device 40 also comprises at least one of an electronic circuit 41, a processor 42, a battery 43, a storage 44, a sensor 45, a communication interface 46 and a proximity sensor 47. With typical examples, the supplementary device 40 is operatively connected or coupled to the interlock 30 of the drive mechanism 11 when attached to the injection device 10.

Additionally or alternatively, the supplementary device 40, particularly its sensor 45 may be operatively connected or coupled to the movable part 26 of the drive mechanism 11. This way the supplementary device 40 is operable to acquire or to obtain information about the momentary status of the drive mechanism 11, which reflects in a position, orientation or movement of the movable part 26. Similarly and as described above in connection with the electronic unit 60 or with the mobile electronic device 100 the supplementary device 40 is operable to wirelessly communicate with a mobile electronic device 80, 100, 120.

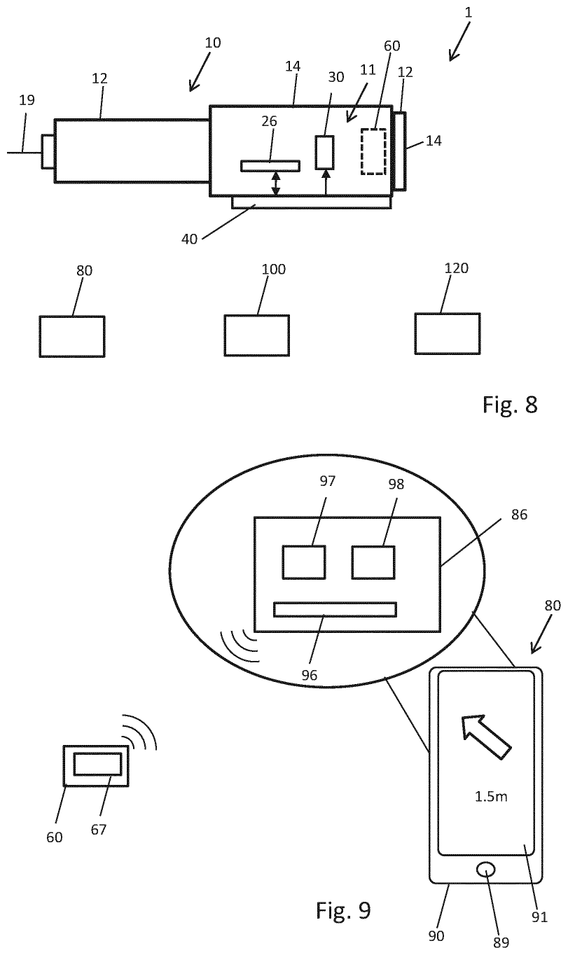

The supplementary device 40 may be also operable to directly communicate with a database 140 via the communication network 130. As further illustrated in FIG. 8, the supplementary device 40 and/or the electronic unit 60 of the injection device 10 may be operable to communicate with at least one or with several of the mobile electronic devices 80, 100, 120. There may be established a wireless communication link between at least two of the electronic unit 60, the supplementary device 40, the mobile electronic device 80, the mobile electronic device 100 and mobile electronic device 120. Moreover, the mobile electronic devices 80, 100, 120 may communicate among each other.

There may be established a first wireless communication link between at least one of the electronic unit 60 and the supplementary device 40 with one of the mobile electronic devices 80, 100, 120. There may be established a further wireless communication link between any of the electronic unit 60, the supplementary device 40, the mobile electronic device 80, 100, 120 with the communication network 130 and/or with the database 140.

The sensor 45, 65, 85, 105, 125 may be implemented as one of an acceleration sensor, a rotation sensor, a position sensor, a distance sensor and a physiologic data capturing sensor of. Implementation of a physiologic data capturing sensor is typically provided with a wearable electronic device 80, 100, 120. By way of the sensor 45, 64, 85, 105, 125 motion data of the respective electronic circuit 41, 61, 81, 101, 121 can be acquired.

Respective motion data can be either stored in the associated storage 44, 64, 84, 104, 124 of the respective electronic circuit 61. The acquired motion data may be also communicated and/or transmitted via the communication interface 46, 66, 86, 108, 126 to another electronic circuit 41, 61, 81, 101, 121. Motion data of a user 4 may be also obtained by establishing a communication link with the database 140 via the network 130. Motion data as collected by any of the sensors 45, 65, 85, 105, 125 may be stored in the database 140 and can be made available to any other electronic circuit 41, 61, 81, 101, 121.

Moreover, at least a portion of the motion data can be acquired or collected by a first mobile electronic device 100, 120, e.g. carried along with the user 4.

A further portion of motion data can be acquired and collected by a sensor 44, 65 attached to or integrated into the injection device 10. Different portions of motion data may be combined and collected through a wireless communication link between respective mobile electronic devices 80, 100, 110 and at least one of the supplementary device 40 and the electronic unit 60. Any of the wireless communication interfaces 46, 66, 86, 106, 126 may be operable to transceive (to transmit and/or to receive) in a specific frequency range that follows a specific transmission protocol. The wireless communication interfaces may comprise multiple communications circuits, each transceiving and in a different frequency range and/or according to a different wireless transmission protocol.

At least one or several of the wireless communication interfaces 46, 66, 86, 106, 126 as described herein comprise at least one RFID-communication circuit (radio frequency identification). The wireless communication interface may comprise an NFC-circuit (near-field communication), e.g. an active NFC-circuit (preferably with associated power source, e.g. a battery such as a printable battery) or a passive NFC-circuit (preferably without a power source powering the circuit). The wireless communication interface may comprise at least one Bluetooth and/or BLE communication circuit (Bluetooth Low Energy). The wireless communication unit may comprise at least one WiFi-communication circuit (wireless fidelity, e.g. according to the IEEE 802.11 standard/protocol). Alternatively or additionally, the wireless communication unit may comprise a magnetometer/compass circuit to detect variation in magnetic fields, e.g. variations produced by a wireless communication circuit of a component of the drug delivery device.

In some implementations, at least one or several of the wireless communication interfaces 46, 66, 86, 106, 126 as described herein are implemented as UWB (ultra wideband) communication interfaces.

At least one of the wireless communication interfaces 46, 66, 86, 106, 126 is configured for generating UWB signals that enable communications between the another one of the wireless communication interfaces 46, 66, 86, 106, 126. The UWB signals include very short RF pulses (e.g., smaller than 1 ns) covering a large portion of the radio spectrum (e.g., bandwidth larger than 500 MHz or 20% of the center frequency, whichever is lower), at a very low energy level. The operating frequency is chosen in accordance with one or more national and federal regulations. For example, a frequency band with wide international acceptance is from about 6.5 GHz to about 8 GHz. In some implementations, the UWB signals include a UWB standard widely accepted and available in smartphones from large vendors such that standard smartphones can be used as the mobile electronic device 80.

In some implementations, the UWB communication includes a proprietary UWB protocol. The proprietary UWB protocol uses an encoding, which consists of a combination of time modulation, signal shape modulation, and amplitude modulation. A proprietary UWB device (e.g. USB dongle for a smartphone) can be used as a wireless tag 120. Transmission of UWB signals may be triggered by a user operation.

In some implementations, and as illustrated in FIG. 9 the wireless communication between at least one of the injection device 10 and the supplementary device 40 with at least one of the mobile electronic devices 80, 100, 120 provides a pager functionality. For instance when a communication link is established by using a UWB communication protocol a precise position tracking of the electronic unit 60 can be provided in principle. Hence, the position of at least one of the drug delivery device 10 and the supplementary device 40 can be precisely determined or measured within a precision of only a few centimeters or even millimeters.

In addition or alternative, the wireless communication link may also provide determination and/or quantitative measuring of at least one of a distance and a relative position between a first electronic circuit and a second electronic circuit, e.g. a distance and those or a relative position between the injection device 10 and one of the mobile electronic devices 80, 100, 120.

As indicated in FIG. 9 the mobile electronic device 80 comprises a communication interface 86 equipped with an antenna 96, a distance unit 97 and a position detection unit 98. The antenna 96, e.g. implemented as a UWB antenna is operable to communicate with the electronic unit 60, alternatively with the electronic circuit 41 of the supplementary device 40. In some implementations, the antenna 96 and hence the communication interface 86 is operable to wirelessly communicate with the communication interface 66 and/or with a proximity sensor 67 of the electronic unit 60. The UWB antenna 96 is not limited to be implemented in the mobile electronic device 80. The antenna 96, the distance unit 97 and the position detection unit 98, configured to generate and/or to process UWB signals for a precise position and distance measurement between any two mobile electronic devices 80, 100, 120 and/or electronic circuit 41, 61 can be likewise implemented in any of the communication interfaces 46, 66, 106, 126.

The antenna 96 may be configured such that the time characteristics of the UWB signals are constant over the frequency spectrum, resulting in minimal pulse distortion. The antenna 96 may exhibit a rather flat frequency spectrum, resulting in wide pulses with minimal resonant distortion. The antenna 96 may be integrated into any one of the communication interfaces 46, 66, 86, 106, 126, typically near a surface of a respective housing to have minimal attenuation of the signal. Possible implementations of the antenna 96 are the integration of a chip antenna, or the integration of a conductive layer acting as the antenna 96 on one of a plastic component of any one of the injection device 10, supplementary device 40, or any one of the mobile electronic device 80, 100, 120. A counterpart communication interface 46, 66, 86, 106, 126 operable to communicate with the antenna 96 may be configured to constantly or intermittently listen for incoming data packets transmitted by the antenna 96.

The distance unit 97 operatively coupled to the antenna 96 may be capable to derive or to quantitatively measure a distance between the electronic unit 60 and the communication interface 86, hence between the electronic unit 60 and the device 80 on the basis of at least one of signal dispersion, time of flight measurement, dynamic or static triangulation or on the basis of any further generally available distance or position measurement scheme being compatible with the respective wireless communication protocol.

The antenna 96 may be implemented as an array antenna being capable to determine a direction from which a signal or a response signal is received from the electronic unit 60. By way of the wireless communication link between the mobile electronic device 80 and one of the electronic unit 60 and the supplementary device 40 not only a distance, such as 1.5 m but also a direction and hence a relative position between the device 10, 40 and 80 can be provided in form of a visible symbol, e.g. on the display 91 of the mobile electronic device 80, thus assisting a user 4 to find or to retrieve the electronic unit 60 in case the respective device should get lost or in case the user should be currently unaware of a place where the injection device 10 can be found.

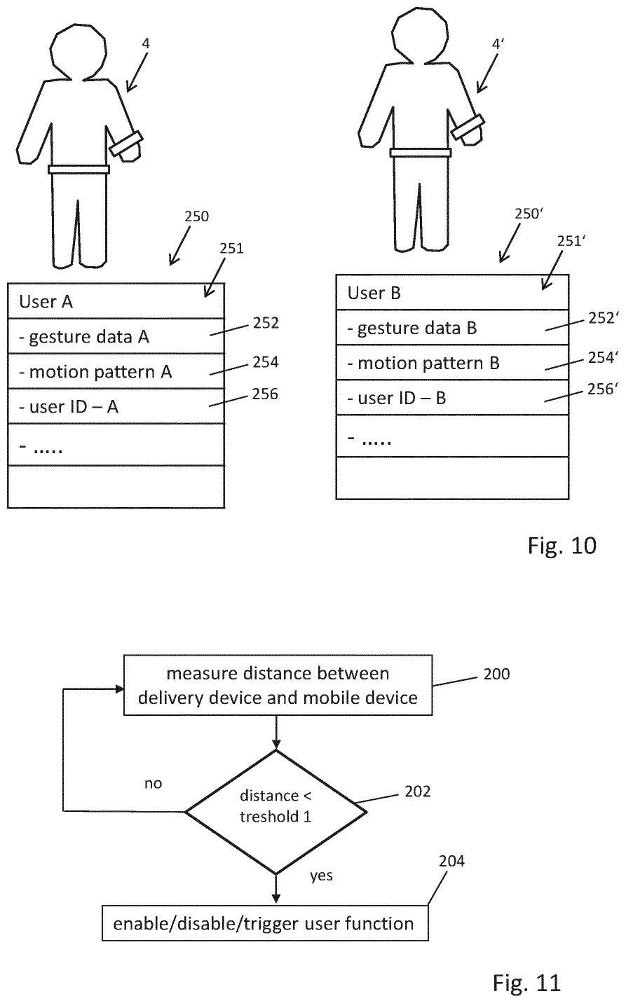

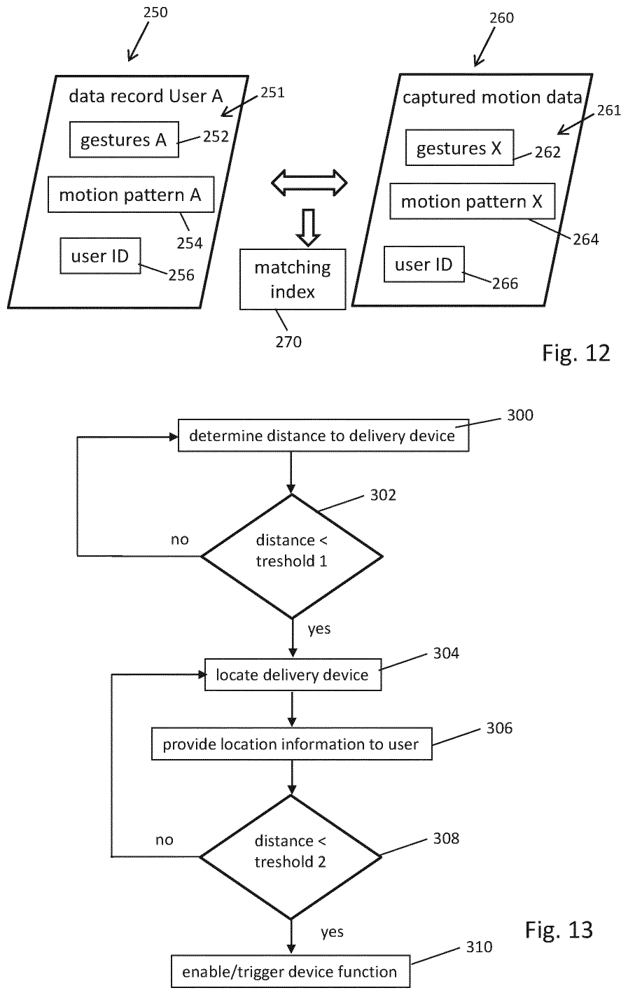

In FIG. 10, a data record 250 of a first person 4 and another data record 250′ of another person 4′ are illustrated. The data records 250, 250′ comprise motion data 251, 251′. Motion data 251 comprises at least one of gesture data 252 and a motion pattern 254 of the user 4. Gesture data 252 characteristic of particular movements or gestures conducted or executed by a user 4, e.g. the way the user walks or the way the user picks up a device.

The motion pattern 254 may be characteristic or indicative of a motion profile of the respective user 4. The motion pattern 254 may contain information about the habits of the user for. E.g. the motion pattern 254 may indicate that a particular user A is located in his home environment at particular days a week and stays there during a specific time interval, e.g. during night times.

The motion pattern 254 may contain a typical movement profile and may provide a mapping of the position of the user over time. Optionally, the data record 250 may also comprise a user identification 256. The user identification 256 may contain a unique identifier of the user. The data record 250 may be subject to a permanent update as the user 4 is equipped with a mobile electronic device 80, 100, 120 being incapable to capture and/or to acquire respective motion data or movement data over time.

The data record 250 comprises motion data 251 and be stored in any of the above-mentioned storages 44, 64, 84, 104, 124. It may be made available upon request, e.g. when the authentication system 1000 conducts a comparison of actually or newly acquired motion data 261 with previously stored motion data 251 in order to determine or to derive a data matching index 270.

As further illustrated in FIG. 10 another user B comprises another data record 250′. Also this data record 250′ may be structured the same way as the data record 250. The data record 250′ also comprises gesture data 252′, a motion pattern 254′ and optionally also a user identification 256′. The motion data 251 is characteristic for a first user 4 while the motion data 251′ is characteristic for another user 4′.

In the flowchart according to FIG. 11 one example of a method of controlling an electronically implemented function of and/or associated with the drug delivery device 1 or injection device 10 is schematically illustrated. In a first step 200 there is measured a distance between the drug delivery device associated or equipped with the electronic circuit 41, 61 as described above and at least one of the mobile electronic devices 80, 100, 120. The wireless communication between the mobile electronic device 80, 100, 120 and the electronic circuit 41, 61 is used to determine or to measure a distance and/or a relative position between the electronic circuit 41, 61 and the mobile electronic device 80, 100, 120.

If the distance is below a predefined approval distance and hence below a respective threshold in step 202, the procedure continues with step 204. For instance, if the distance is below a predefined threshold distance, e.g. below a predefined approval distance the electronically implemented function of the electronic circuit 41, 61 the electronically implemented function of the drug delivery device 1 will be one of enabled, disabled, triggered or terminated by the processor 42, 62. In some implementations, the electronically implemented function of the electronic circuit 41, 61 is operable to interlock the drive mechanism 11 of the injection device 10. Then and with the distance measurement the electronic circuit 41, 61 may be operable to unlock or to release the interlock 30 depending on the size of the distance measured between the mobile electronic device 80, 100, 120 and one of the electronic circuit 41, 61 of one of the drug delivery device 1 or supplementary device 40 attached to the injection device 10.

If in step 202 it is determined that the distance as measured by way of the wireless communication is above the predefined threshold the procedure will returns to step 200 and repeatedly measure the distance between the drug delivery device 1 and the mobile electronic device 80, 100, 120.

With the further example as illustrated in FIG. 13 in a first step 300 a distance between the electronic circuit 41, 61 integrated into or attached to the drug delivery device 1 is measured and/or determined. In a subsequent step 302 the distance is evaluated or analyzed. There, it is determined if the distance is below a predefined first threshold. If the evaluation in step 202 reveals that the distance is below a first predefined distance threshold the procedure continues with step 304. If in step 302 it is determined that the distance is above the predefined first threshold the procedure returns to step 300.

In step 304 and hence when the distance is within a predefined range the wireless communication between the communication interface 46, 66 of the electronic circuit 41, 61 and the mobile electronic device 80, 100, 120 is further used to locate the injection device and/or the mobile electronic device. Here, a relative position between the electronic circuit 41, 61 and the mobile electronic device 80, 100, 120 is determined or measured.

The relative position is indicative of a vector and is hence indicative of a direction of the shortest distance between the electronic circuit 41, 61 attached to or integrated into the drug delivery device 1 and the mobile electronic device 80, 100, 120. Once the relative position has been determined or calculated on the basis of the exchanged wireless signals between the communication interface 46, 66 and a counterpart or mobile communication interface 86, 106, 126 the respective position information or location information is provided to a user of the mobile electronic device 80, 100, 120 and/or to a user 4 of the drug delivery device 1.

The information may be provided to the user 4 e.g. by way of a signaling unit 88, 108, 128 operable to generate a user-perceivable signal allowing a user to identify and/or to find the drug delivery device equipped with the electronic circuit 41, 61. Here, the wireless communication between the electronic circuit 41, 61 and the mobile electronic device 80, 100, 120 can be used to provide a kind of an electronic pager function, thereby assisting a user in finding the drug delivery device in case it got lost or in case a user is unaware, where the injection device 10 or drug delivery device 1 has been stored or used recently.

Optionally and in a further step 308 there is conducted a further distance measurement through wireless communication between we the electronic circuit 41, 61 and the mobile electronic device 80, 100, 120. Here it may be determined if the distance is below a second distance threshold. If it should be determined in step 308, that the distance is below the second predefined minimum threshold the procedure may continue with step 310, thereby approving access to an electronically implemented function of the electronic circuit 41, 61 or electronic function of the drug delivery device 1.

Such a twofold distance measurement or distance evaluation in steps 302, 308 is beneficial in that general authorization of a user can be already provided when the user with his mobile electronic device 80, 100, 120 is in a predefined spatial vicinity of the drug delivery device 1. If the distance between the mobile electronic device 80, 100, 120 is within a first distance or range a general user authentication procedure can be executed, which due to the limited distance between the devices may only consume a limited or reduced amount of electric energy compared to a situation, in which an authentication routine is conducted at a comparatively large distance between the mobile electronic device and the drug delivery device 1.

In order to finally approve execution of the electronically implemented function it is intended that the distance is within a second, i.e. smaller range. In this way and when the mobile electronic device 100, 120 is e.g. implemented as a wearable electronic device, such as a smartwatch, it can be controlled if the user holds the drug delivery device in a correct manner. For instance, and on the basis of this second distance check as conducted in step 308 it may be measured or detected if an injection device 10 is held in the same hand of a user 4 to which also a wearable electronic device 100, 120 is attached to. This way, patient safety as well as compliance with an intended or prescribed use of the injection device can be enhanced.

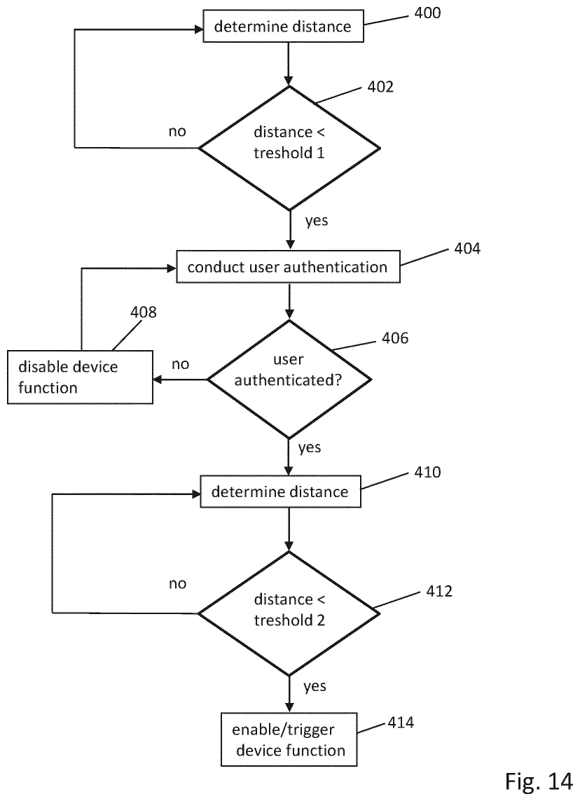

In the further example according to FIG. 14 the procedure of controlling the electronically implemented function of or associated with the drug delivery device 1 commences with a first step 400, in which a distance between the electronic circuit 41, 61 and the mobile electronic device 80, 100, 120 is measured or determined. In a subsequence step 402 it is checked or evaluated if the measured or determined distance is below a predefined first distance threshold. In case the distance is above the threshold the procedure returns to step 400. If in step 402 it should be determined that the distance is below the first distance threshold the procedure continues with step 404. In step 404, the mobile electronic device 80, 100, 120 and/or at least one of the electronic circuits 41, 61 is operable or is triggered to conduct a user authentication procedure as described above.

User authentication may be based on receiving or exchanging and evaluating of an identification signal. Alternatively or additionally, the user can be identified by conducting, analyzing or by evaluating motion data of a user as described above.

In a subsequent step 406 it is determined if the user 4 is authorized to use the drug delivery device 1. If in step 406 user authentication should fail a device function, e.g. an electronically implemented function of the electronic circuit 41, 61 of one of the delivery device 1, injection device 10 or supplementary device 40 may be disabled. The procedure may then return to step 404.

If user authentication in step 406 is approved the procedure continues with step 410. Here, the distance between the mobile electronic device 80, 100, 120 and the electronic circuit 41, 61 is determined or measured again. Then, and in a further step 412 there is conducted a further distance measurement through wireless communication between the electronic circuit 41, 61 and the mobile electronic device 80, 100, 120. Here it may be determined if the distance is below a second distance threshold. If it should be determined in step 412, that the distance is below the second predefined minimum threshold the procedure may continue with step 414, thereby approving access to an electronically implemented function of the electronic circuit 41, 61 or electronic function of the drug delivery device 1. Here, the second distance measuring as conducted by the steps 410, 412 may closely correspond to the distance measuring as described above in connection with steps 304 and 308 of FIG. 13.

### REFERENCE NUMBERS

1 drug delivery device
2 distal direction
3 proximal direction
4 user
5 medicament container
6 stopper
7 seal
8 medicament
10 injection device
11 drive mechanism
12 dose dial
14 dose button
15 inner body
16 housing
17 protective cap
18 container housing
19 injection needle
20 window
21 number sleeve
22 drive sleeve
24 piston rod
25 pressure piece
26 movable part
30 interlock
supplementary device
41 electronic circuit
42 processor
43 battery
44 storage
45 sensor
46 communication interface
47 proximity sensor
48 signaling unit
49 fastener
50 housing
51 display
60 electronic unit
61 electronic circuit
62 processor
63 battery
64 storage
65 sensor
66 communication interface
67 proximity sensor
68 signaling unit
80 mobile electronic device
81 electronic circuit
82 processor
83 battery
84 storage
85 sensor
86 communication interface
87 proximity sensor
88 signaling unit
89 input
90 housing
91 display
95 satellite-based positioning system
96 antenna
97 distance unit
98 position detection unit
100 mobile electronic device
101 electronic circuit
102 processor
103 battery
104 storage
105 sensor
106 communication interface
107 proximity sensor
108 signaling unit
110 housing
111 display
112 strap
120 mobile electronic device
121 electronic circuit
122 processor
123 battery
125 sensor
126 communication interface
127 proximity sensor
128 signaling unit
129 housing
130 network
131 access point
140 database
250 data record
251 motion data
252 gesture data
254 motion pattern
256 user identification
260 data record
261 motion data
262 gesture data
264 motion pattern
266 user identification
270 matching index
1000 authentication system

1.-17. (canceled)

18. An electronic circuit for a drug delivery device, the drug delivery device being operable to dispense a medicament, the electronic circuit comprising:
a communication interface configured to wirelessly communicate with a mobile electronic device and to transmit at least a wireless signal to the mobile electronic device, the wireless signal indicating at least one of a distance or a relative position between the electronic circuit and the mobile electronic device; and
a processor operatively connected to the communication interface and configured to enable, to disable, to trigger, or to terminate execution of an electronically implemented function of the electronic circuit based on an analysis of the at least one of the distance or the relative position.

19. The electronic circuit according to claim 18, wherein the electronic circuit is configured to communicate with the mobile electronic device to quantitatively measure the distance between the electronic circuit and the mobile electronic device.

20. The electronic circuit according to claim 19, wherein processor is configured to disapprove access to the electronically implemented function of the electronic circuit if the distance is above a predefined approval distance.

21. The electronic circuit according to claim 18, wherein the processor is further configured to analyze at least one of the distance or the relative position.

22. The electronic circuit according to claim 18, further comprising a proximity sensor operatively connected to the processor and configured to measure at least one of the distance or the relative position between the electronic circuit and the mobile electronic device.

23. The electronic circuit according to claim 22, wherein at least one of the communication interface or the proximity sensor is implemented as an ultra-wideband (UWB)-based interface or an UWB-based sensor, respectively, and is configured to generate, to transmit, to receive and/or to process electromagnetic ultra-wideband signals.

24. The electronic circuit according to claim 18, wherein the processor is operable to enable or to trigger the electronically implemented function only when:
the distance between the electronic circuit and the mobile electronic device is within a predefined operating distance, and/or
the electronic circuit is within a predefined relative operating position with regard to mobile electronic device.

25. The electronic circuit according to claim 18, wherein the processor is configured to generate a control command based on the analysis of at least one of the distance or the relative position, the control command being configured to cause at least one of:
generating a user-perceivable alert or signal by a signaling unit,
locking or unlocking of a drive mechanism of the drug delivery device by an electromechanical interlock,
triggering or terminating transmission of data by the communication interface, or switching the electronic circuit from a standby mode into an active mode, or vice versa.

26. The electronic circuit according to claim 18, wherein the communication interface is configured to exchange user specific data of a user with the mobile electronic device, wherein the user specific data specify at least one of:
a medicament type,
a time and/or a date of a medicament dispensing action,
an amount of dispensed medicament, or
motion of the user.

27. A drug delivery device operable to dispense a medicament, the drug delivery device comprising:
a housing to accommodate a medicament container filled with the medicament;
a drive mechanism operatively connectable to the medicament container to expel or to withdraw the medicament from the medicament container; and
an electronic circuit integrated into the housing and/or operatively connected to the drive mechanism, wherein the electronic circuit comprises:
a communication interface configured to wirelessly communicate with a mobile electronic device and to transmit at least a wireless signal to the mobile electronic device, the wireless signal indicating at least one of a distance or a relative position between the electronic circuit and the mobile electronic device; and
a processor operatively connected to the communication interface and configured to enable, to disable, to trigger, or to terminate execution of an electronically implemented function of the electronic circuit based on an analysis of the at least one of the distance or the relative position.

28. A supplementary device for a drug delivery device, the supplementary device comprising:
a housing;
a fastener for fastening the supplementary device to a housing of the drug delivery device; and
an electronic circuit, wherein the electronic circuit comprises:
a communication interface configured to wirelessly communicate with a mobile electronic device and to transmit at least a wireless signal to the mobile electronic device, the wireless signal indicating at least one of a distance or a relative position between the electronic circuit and the mobile electronic device; and
a processor operatively connected to the communication interface and configured to enable, to disable, to trigger, or to terminate execution of an electronically implemented function of the electronic circuit based on an analysis of the at least one of the distance or the relative position.

29. A method of controlling an electronically implemented function of or associated with a drug delivery device, the method comprising:
establishing of a wireless communication link between an electronic circuit attached to or integrated into the drug delivery device and a mobile electronic device;
determining at least one of a distance or a relative position between the electronic circuit and the mobile electronic device through transmission of wireless signals between the electronic circuit and the mobile electronic device;
analyzing or evaluating at least one of the distance or the relative position; and
at least one of enabling, disabling, triggering or terminating execution of an electronically implemented function of at least one of the electronic circuit or the mobile electronic device based on analyzing or evaluating of the at least one of the distance or the relative position.

30. The method according to claim 29, further comprising:
quantitatively measuring the distance between the electronic circuit and the mobile electronic device.

31. The method according to claim 29, further comprising:
disapproving access to the electronically implemented function of the electronic circuit if the distance between the electronic circuit and the mobile electronic device is above a predefined approval distance.

32. The method according to claim 29, further comprising:
measuring at least one of the distance or the relative position between the electronic circuit and the mobile electronic device by using a proximity sensor operatively connected to the processor.

33. The method according to claim 32, wherein measuring at least one of the distance or the relative position between the electronic circuit and the mobile electronic device comprises generating, transmitting, receiving and/or processing electromagnetic ultra-wideband signals.

34. The method according to claim 29, further comprising:
enabling or triggering the electronically implemented function only when:
the distance between the electronic circuit and the mobile electronic device is within a predefined operating distance, and/or
the electronic circuit is within a predefined relative operating position with regard to mobile electronic device.

35. The method according to claim 29, further comprising:
generating a control command based on the analysis of at least one of the distance or the relative position, the control command being configured to cause at least one of:
generating a user-perceivable alert or signal by a signaling unit,
locking or unlocking of a drive mechanism of the drug delivery device by an electromechanical interlock,
triggering or terminating transmission of data by the communication interface, or
switching the electronic circuit from a standby mode into an active mode, or vice versa.

36. The method according to claim 29, further comprising:
exchanging user specific data of a user between the communication interface and the mobile electronic device, wherein the user specific data specify at least one of:
a medicament type,
a time and/or a date of a medicament dispensing action,
an amount of dispensed medicament, or
motion of the user.
